(19) United States
(12) Patent Application Publication
(10) Pub. No.: US 2007/0055113 A1
(43) Pub. Date: Mar. 8, 2007

(54) Method and apparatus for homeopathic provings and diagnosis

(76) Inventor: Michael J. Quinn, San Rafael, CA (US)

Correspondence Address: JOHNSON & STAINBROOK, LLP, 3558 ROUND BARN BLVD., SUITE 203, SANTA ROSA, CA, 95403, US

(21) Appl. No.: 10/558,144

(22) PCT Filed: May 20, 2004
(86) PCT No.: PCT/US04/15933

§ 371 Date: Nov. 21, 2005

Related U.S. Application Data
(60) Provisional application No. 60/472,363, filed on May 20, 2003.

(51) Int. Cl. A61K 36/18; A61B 5/00; A61K 33/00

(52) U.S. Cl. 600/300; 424/600; 424/725

(57) ABSTRACT

An improved method and apparatus for homeopathic provings and diagnosis includes a method of “proving” home-opathic medicines and diagnosing patients by recording one or more physiological, psychological or mental processes of the prover exposed to controlled and standardized stimuli when he or she is experiencing the effects of the homeopathic proving, and then recording from patients the same one or more physiological, psychological or mental processes during exposure to the same stimuli and comparing the recordings, or “symptoms,” of the provers and patients to find a good match and therefore the correct medicine for that patient.

### BACKGROUND OF TIHE INVENTION

1. Technical Field

The present invention relates generally to homeopathic medicine, and more particularly to an improved method and apparatus for homeopathic provings and diagnosis.

2. Background Art

Homeopathy is a branch of medicine that has existed since 1810. The fundamental principle of Homeopathy provides, in part, that a sick person (a patient) can be successfully treated with a substance that will cause in healthy people the very same set of symptoms that the patient is suffering from. This is known as “The Law of Similars.” The second principle is that the patient should be given the “minimum dose” necessary to bring about improvement in the patient's symptoms or cure. As an example, a patient suffering from insomnia might be prescribed coffee by his or her homeopath if the patient's symptoms of insomnia matches the set of symptoms suffered by people who are kept awake at night after drinking too much coffee late in the day. However, it is not usually therapeutically efficacious to provide a patient with a cup of coffee. Instead, low doses are typically prescribed, and the insomniac will be cured of his or her insomnia if the diagnosis is correct and if the correct dose of coffee is administered.

The homeopathic diagnosis of the patient has as its goal the discovery of a substance that causes in healthy people the same symptoms as the patient is experiencing. The homeopathic diagnosis consists of the process of eliciting from the patient a description of the patient's symptoms and then matching these symptoms with the symptoms of known substances that provoke symptoms in healthy people. A practitioner of homeopathy will coordinate the process using his/her knowledge and informational resources, such as books and automated data processing and computing systems, to identify the best match possible between the symptoms of the patient and the known sets of symptoms of substances databases.

Further principles of Homeopathy state that the set of a patient's symptoms is a subset of the symptoms caused by the substance. All of the patient's symptoms should be known to be symptoms caused by a particular substance. Not all of the known symptoms of the substance need be present in the patient to make a good match. Occasionally a good match is made even when the patient has symptoms that are not currently known to be symptoms caused by the substance. If the substance proves curative to the patient, and if the symptom hitherto unknown to be a symptom of the substance is also eliminated, then that symptom may be deduced to be a symptom of the substance as well.

The great body of Homeopathic knowledge of symptoms of substances was created by several methods. Some, but not all, of these methods were provings, cured symptoms, and observation of poisonings. Provings are Homeopathic research processes in which generally healthy people (“provers”) ingest an intoxicating but nonlethal dose of a substance and record their symptoms. Provings may be solitary in which the person acts alone; or witnessed in an unblinded manner, where the prover and the witness know what substance the prover is taking; or in a single blinded manner where only the witness knows what the substance is; or in a double blinded manner where neither the prover nor the witness knows the identity of the substance. Cured symptoms are added to the collective databases as described above, and poisoning symptoms may be added from a review of the medical toxicological literature. This is usually not as helpful because the medical literature does not record the symptoms in sufficient detail and seldom includes the modalities of the symptoms, that is, the way the symptoms are increased or decreased as the patient's circumstances change.

The current state of the art of Homeopathic diagnosis relies upon the patient's verbal description of his/her symptoms and the manual recording of the patient's symptoms by the homeopath. The knowledge and skill of the homeopath are based on his/her ability to elicit good report of symptoms from the patient and to match the patient's symptoms with the symptoms recorded in homeopathic resources. This is a complex and difficult process, as any experienced homeopath will readily admit.

Currently there are programs operating on automated computing devices that assist the homeopath in searching for a good match between the symptoms of the patient and the database of proving and cured symptoms. However, the limitations in practicing the method of the current state of the art are manifold. These limitations include, but are not limited to, the amount of time available to elicit the patient's symptoms and analyze the symptoms, an incomplete knowledge of the symptoms caused by substances, an incomplete knowledge on the part of the homeopath of the known symptoms of substances, an incomplete and sometimes inaccurate listing of the known symptoms of substances in books or electronic data processing systems data storage devices, an incomplete questioning of the patient to determine his/her symptoms, an incomplete recall and description of his/her symptoms by the patient, and an inability on the part of the patient to know himself/herself well enough to recognize and discern changes in sensation, mood, affect, perception, and bodily condition, and then to accurately report on his/her observations. This is in part due to social conditioning that might make difficult the admission of certain socially undesirable behavior, thoughts, symptoms, feelings, urges, or desires.

There are further limitations in practicing the method of the current art, including the rapidly evolving availability of truly novel and exotic experiences, as many new experiences may not be in the symptoms of substances databases. For example a person who experiences specific symptoms from rocket travel to the moon will be hard pressed to find many similar symptoms listed in symptoms databases developed from earthbound people.

Furthermore, the amount of information transmitted from patient to homeopath in the diagnostic session is limited. Typically the homeopath will spend from several minutes to several hours interviewing the patient in order to elicit an accurate report of his/her symptoms. The demands of time and conventional human activity necessitates a limited interaction. During a one hour interview, for example, the number of symptoms a patient may relate at a normal conversational rate of speech is limited to several hundred symptoms at most and typically is much less. If the complete description of a symptom with its modalities lasted about one minute for an average patient, then only 60 symptoms could be elicited in one hour.

The most commonly used symptom databases have hundreds of thousands of symptoms listed for several thousand substances. Thus, the likelihood of finding a match between the patient's symptoms and the recorded symptoms of substances in several hours time is remote. Those examples of patients cured by a homeopath's ability to make the correct match and find the correct substance that cures the patient are a testimony to the skill and knowledge of the homeopath. The homeopath is, of course, taking in information every second about the patient as they sit together, typically with a desk between them, in the homeopath's office. The astute homeopath learns a great deal about the patient from this observation of the patient and many times finds this information as useful or more useful than the symptoms the patient relates. The astute homeopath also guides the recitation or report of symptoms and, as any good detective, adapts to the clues and continuously evaluates and changes paths on his/her investigation while holding several lines of investigation open at any moment. The astute homeopath, however, has a hard time transferring this skill to the novice homeopath.

The skill and knowledge level of homeopaths is limited. If all homeopaths had the skill and knowledge level of concert pianists, many more patients could be cured. Yet there would still be a limitation on the rate of transmission of symptoms from patient to homeopath. The present invention seeks to overcome that limitation by the use of modem technology, thereby making accurate homeopathic diagnosis a more common and easily achieved event to the benefit of many suffering patients.

The foregoing discussion reflects the current state of the art of which the present inventor is aware. Reference to, and discussion of, this information is intended to aid in discharging Applicant's acknowledged duty of candor in disclosing information that may be relevant to the examination of claims to the present invention. However, it is respectfully submitted that none of the prior art references disclose, teach, suggest, show, or otherwise render obvious, either singly or when considered in combination, the invention described and claimed herein.

### DISCLOSURE OF INVENTION

The present invention provides an improved method and apparatus for homeopathic provings and diagnosis, and consists of a new method of “proving” homeopathic medicines and diagnosing patients by recording one or more physiological, psychological or mental processes of the prover exposed to controlled and standardized stimuli when he or she is experiencing the effects of the homeopathic proving, and then recording from patients the same one or more physiological, psychological or mental processes during exposure to the same stimuli and comparing the recordings, or “symptoms,” of the provers and patients to find a good match and therefore the correct medicine for that patient.

It is therefore an object of the present invention to provide a new and improved method to increase the speed of the transmission of the patient's symptoms.

It is another object of this invention to increase the number of symptoms that can be elicited and matched against the known database of provers' symptoms. If ten points of data were to be recorded each second, which is easily within the capabilities of current automated single or multi-channel recording equipment, then, since there are sixty seconds per minute and sixty minutes per hour, or 3,600 seconds per hour, 36,000 data points might be recorded in one hour. It is possible with technical improvements that one million data points could be recorded.

It is thus yet another object of this invention that advanced automated pattern recognition programs operating on automated computing devices match the recordings of the patient against the stored recordings of thousands of provers who ingested hundreds or thousands of substances. The likelihood of finding a good match increases as the number of data points increases for both the patient and prover recordings.

Other novel features which are characteristic of the invention, as to organization and method of operation, together with further objects and advantages thereof will be better understood from the following description considered in connection with the accompanying drawing, in which preferred embodiments of the invention are illustrated by way of example. It is to be expressly understood, however, that the drawing is for illustration and description only and is not intended as a definition of the limits of the invention. The various features of novelty which characterize the invention are pointed out with particularity in the claims annexed to and forming part of this disclosure. The invention resides not in any one of these features taken alone, but rather in the particular combination of all of its structures for the functions specified.

There has thus been broadly outlined the more important features of the invention in order that the detailed description thereof that follows may be better understood, and in order that the present contribution to the art may be better appreciated. There are, of course, additional features of the invention that will be described hereinafter and which will form additional subject matter of the claims appended hereto. Those skilled in the art will appreciate that the conception upon which this disclosure is based readily may be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the Abstract is to enable the national and regional patent office(s) and the public generally, and especially the scientists, engineers and practitioners in the art who are not familiar with patent or legal terms or phraseology, to determine quickly from a cursory inspection the nature and essence of the technical disclosure of the application. The Abstract is neither intended to define the invention of this application, which is measured by the claims, nor is it intended to be limiting as to the scope of the invention in any way.

Certain terminology and derivations thereof may be used in the following description for convenience in reference only, and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a block diagram illustrating the method and apparatus for homeopathic provings of this invention; and

FIG. 2 is a block diagram illustrating the method and apparatus for homeopathic diagnosis of this invention.

### BEST MODE FOR CARRYING OUT THE INVENTION

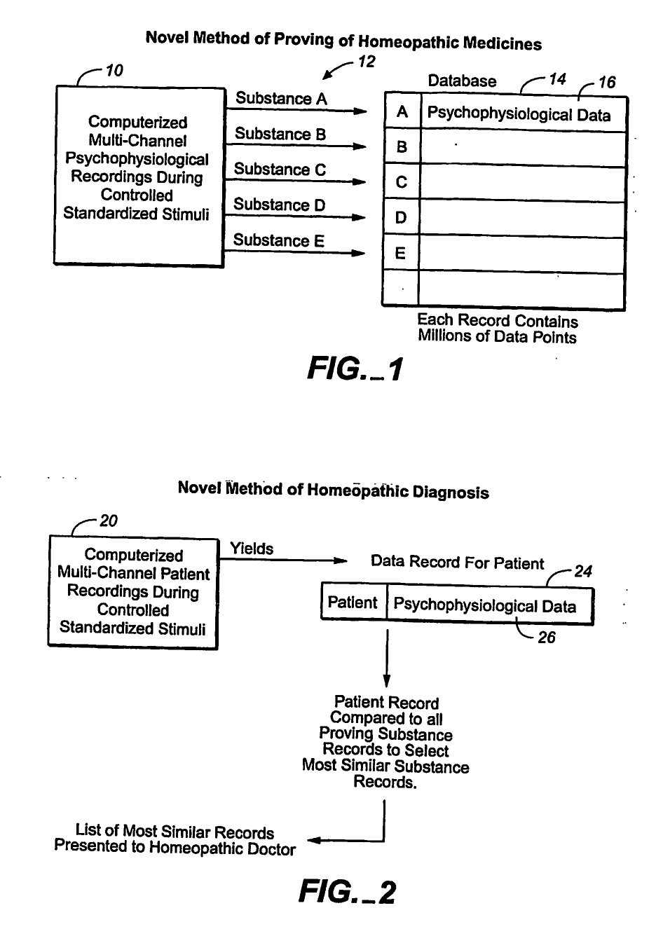

FIG. 1 is a block diagram illustrating the method and apparatus for homeopathic provings of this invention. Recording device 10 (such as an analog or digital high speed single or multi-channel automated recording system) measures the prover's psychophysiological responses to stimuli while experiencing the effect of one or more substances 12, and delivers that data to a data processing system 14 with data storage capability to generate a psychophysiological database 16 of symptoms produced by substances 12. Each record may contain millions of data points.

Provers are thus connected to the automated recording system that monitors and records one or more physiological, psychological or mental processes, such as but not limited to, heart rate, respiratory rate, electroencephalographic patterns, and so forth, during the proving process. Recordings are made of the provers' responses to standardized and controlled stimuli (e.g., lights, sounds, smells, tastes, touch, movies or other audiovisual works, or other stimulus) during the period of time in which they are experiencing symptoms of the proving. These responses are collected from one or more provers for each substance and analyzed using automated data processing equipment and/or processes.

FIG. 2 is a block diagram illustrating the method and apparatus for homeopathic diagnosis of this invention. When a patient approaches a homeopath for assistance in reducing his/her symptoms, the homeopath diagnoses the patient by connecting the patient to the same or analogous recording equipment 20 and recording the patient's physiological, psychological or mental processes responses to the same controlled and standardized stimuli to which the provers were exposed, delivering the data to the same or analogous data processing system 24 to generate a patient psychophysiological database 26.

The patient's automated recordings 26 of responses to stimuli are then compared to the prover's automated recordings 16 of responses to the same stimuli in order to make a match between the recorded responses, or symptoms, of the provers and the recorded responses, or symptoms, of the patient, and select the most similar substance records. The matching analysis may be performed manually or may be performed by programmable data processing devices, wherein the list of the most similar records is presented to the homeopathic doctor for evaluation.

The foregoing disclosure is sufficient to enable one having skill in the art to practice the invention without undue experimentation, and provides the best mode of practicing the invention presently contemplated by the inventor. While there is provided herein a full and complete disclosure of the preferred embodiments of this invention, it is not intended to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Accordingly, the proper scope of the present invention should be determined only by the broadest interpretation of the appended claims so as to encompass all such modifications as well as all relationships equivalent to those illustrated in the drawings and described in the specification.

What is claimed as invention is:

1. A method for proving homeopathic medicines, said method comprising the steps of:
providing a recording device to measure a prover's psychophysiological responses to a stimulus while experiencing the effect of a substance; and
delivering those measurements to a data processing system to generate a psychophysiological database of symptoms produced by the substance.

2. The method for proving homeopathic medicines of claim 1 wherein said psychophysiological responses comprise heart rate.

3. The method for proving homeopathic medicines of claim 1 wherein said psychophysiological responses comprise respiratory rate.

4. The method for proving homeopathic medicines of claim 1 wherein said psychophysiological responses comprise electroencephalographic patterns.

5. The method for proving homeopathic medicines of claim 1 wherein said psychophysiological responses comprise mental processes.

6. A method for homeopathic diagnosis comprising the steps of:
providing a recording device to measure a patient's psychophysiological responses to a stimulus;
delivering those measurements to a data processing system to generate a patient psychophysiological database.

7. The method for homeopathic diagnosis of claim 6 further including the steps of:
providing a recording device to measure a prover's psychophysiological responses to the stimulus while experiencing the effect of a substance;
delivering those measurements to a data processing system to generate a prover's psychophysiological database of symptoms produced by the substance; and
comparing the patient psychophysiological database to the prover's psychophysiological database.

8. The method for homeopathic diagnosis of claim 6 wherein said psychophysiological responses comprise heart rate.

9. The method for homeopathic diagnosis of claim 6 wherein said psychophysiological responses comprise respiratory rate.

10. The method for homeopathic diagnosis of claim 6 wherein said psychophysiological responses comprise electroencephalographic patterns.

11. The method for homeopathic diagnosis of claim 6 wherein said psychophysiological responses comprise mental processes.
